(19) United States
(12) Patent Application Publication
(10) Pub. No.: US 2017/0344701 A1
(43) Pub. Date: Nov. 30, 2017

(54) IMAGING METHOD FOR CARRYING OUT A MEDICAL EXAMINATION

(71) Applicant: Siemens Healthcare GmbH, Erlangen (DE)

(72) Inventor: Thomas ALLMENDINGER, Forchheim (DE)

(73) Assignee: Siemens Healthcare GmbH, Erlangen (DE)

(21) Appl. No.: 15/596,035

(22) Filed: May 16, 2017

(30) Foreign Application Priority Data
May 24, 2016 (DE) 102016209032.1

(51) Int. Cl. G06F 19/00; A61B 6/03; A61B 5/055

(52) U.S. Cl. CPC G06F 19/321; G06F 19/345; A61B 5/055; A61B 6/032

(57) ABSTRACT

A method and a system are disclosed for carrying out an imaging medical examination. In an embodiment, the method includes: creation of a decision tree on the basis of training data sets, each including data on patient properties and an assigned scan protocol; selection of a scan protocol based on the decision tree created and a patient data set including data on the patient properties of the patient to be examined; and creation of an image via an imaging device based on the selected scan protocol.

### PRIORITY STATEMENT

The present application hereby claims priority under 35 U.S.C. §119 to German patent application number DE 102016209032.1 filed May 24, 2016, the entire contents of which are hereby incorporated herein by reference.

### FIELD

At least one embodiment of the present invention generally relates to a method and/or an imaging device for carrying out an imaging examination.

### BACKGROUND

When operating a CT scanner, a clinical requirement is used as the basis for the selection of a scan protocol defining all technical parameters for the creation of CT images. However, it may be necessary to adapt some of these parameters on the basis of individual patient properties.

The most frequent patient properties necessitating an adaptation of this kind include the weight and height of the patient, the ability to hold breath, general readiness to cooperate and, in the case of cardiovascular examinations, the heart rate and the heart rhythm.

Such adaptations of the scan protocol can be complicated so that frequently they cannot be implemented optimally when operational. As a result, the adaptation of the scan protocol is preferably automated. During such an automated adaptation of the scan protocol to a specific patient, it is important that the reasons for a change to the parameters of the scan protocol be intelligible to an operator of the CT scanner. In particular in the case of CT scanners, it is not possible, to carry out a plurality of examinations with different scan protocols in order then to select the best possible image since the patient is exposed to radiation during each of these examinations. Before the operator performs a scan, the operator should be convinced of the plausibility of the parameters of the scan protocol.

This can be resolved in that the automatic algorithms used to determine the scan protocol are kept simple enough to ensure intelligibility. Complex high-grade non-linear decision-making procedures, such as, for example, neuronal networks, do not enable an operator to identify the reason why a parameter of the scan protocol has been changed by the algorithm. On the other hand, excessive simplicity restricts the image quality of the imaging method and also the methods used for training or learning with respect to the imaging method.

A further approach entails precise documentation and extensive training with respect to a complex algorithm, such as, for example, dose regulation. However, the inherent complexity means the individual configuration is difficult to adjust.

### SUMMARY

At least one embodiment of the present invention defines a scan protocol for performing a patient-specific imaging medical examination such that good image quality is achieved and the parameter changes made are intelligible.

Advantageous embodiments are the subject matter of the claims, the description and the figures.

A first embodiment is directed to a method for carrying out an imaging medical examination with the following steps: creation of a decision tree on the basis of training data sets each of which comprises data on patient properties and an assigned scan protocol; selection of a scan protocol based on the decision tree created and a patient data set comprising data on the patient properties of the patient to be examined; and creation of an image via an imaging device based on the selected scan protocol. This, for example, achieves the technical advantage that a scan protocol is selected with which high image quality is achieved in an intelligible way.

A second embodiment is directed to an imaging system for carrying out a medical examination with: a decision-tree creator for the creation of a decision tree on the basis of training data sets each of which comprises data on patient properties and an assigned scan protocol; a selecting facility for the selection of a scan protocol based on the decision tree created and a patient data set comprising data on the patient properties of the patient to be examined; and an imaging device for the creation of an image via the imaging device based on the selected scan protocol. The imaging system achieves the same technical advantages as those achieved by the method according to the first embodiment.

An embodiment is directed to a method for creating an image related to a patient to be examined during a medical examination, comprising: creating of a decision tree based upon training data sets, each of the training data sets including data on patient properties and an assigned scan protocol; selecting a scan protocol based on the decision tree created and a patient data set including data on patient properties of the patient to be examined; and creating the image, via an imaging device, based on the selected scan protocol.

An embodiment is directed to a n imaging system for creating an image related to a patient to be examined during a medical examination, comprising: an imaging device; a first memory storing computer-readable instructions; and one or more processors. The one or more processors are configured to execute computer-readable instructions to create a decision tree based upon training data sets, each of the training data sets including data on patient properties and an assigned scan protocol, select a scan protocol based on the decision tree created and a patient data set including data on patient properties of the patient to be examined, and create the image, via the imaging device, based on the selected scan protocol.

Another embodiment is directed to a non-transitory computer program product or computer readable medium comprising program sections or software code sections, directly loadable into the memory of a digital computer, to carry out the method according to the first embodiment when the program sections or software code sections are executed by the computer.

The computer program product or computer readable medium can be formed by a computer program or comprise at least one additional component in addition to the computer program. The at least one additional component can be embodied as hardware and/or software.

One example of the at least one additional component, which is embodied as hardware, is a computer readable storage medium that can be read by the digital computer and/or on which the software code sections are stored.

One example of the at least one additional component, which is embodied as software, is a cloud application program embodied to divide the software code sections into different processing units, in particular different computers, of a cloud computing system, wherein each of the processing units is embodied to execute one or more software code sections.

In particular, the software code sections can be used to carry out the method according to the first aspect when the software code sections are executed by the processing units of the cloud computing system.

### BRIEF DESCRIPTION OF THE DRAWINGS

Example embodiments the invention are shown in the drawing and described in more detail below. The drawings show:

FIG. 1 a schematic representation of an imaging system;

FIG. 2 a schematic creation of a decision tree;

FIG. 3 a scatter plot with patient properties;

FIG. 4 an automatically created result of a decision tree for training data; and

FIG. 5 a block diagram of a method for carrying out an imaging medical examination.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or porcessors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (procesor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

A first embodiment is directed to a method for carrying out an imaging medical examination with the following steps: creation of a decision tree on the basis of training data sets each of which comprises data on patient properties and an assigned scan protocol; selection of a scan protocol based on the decision tree created and a patient data set comprising data on the patient properties of the patient to be examined; and creation of an image via an imaging device based on the selected scan protocol. This, for example, achieves the technical advantage that a scan protocol is selected with which high image quality is achieved in an intelligible way.

In one technically advantageous embodiment of the method, the decision tree is created on the basis of a C4.5 algorithm and/or an ID3 algorithm. This, for example, achieves the technical advantage that the decision tree can be created with little effort.

In a further technically advantageous embodiment of the method, the decision tree is created on the basis of the averaging of a plurality decision trees. This, for example, achieves the technical advantage of improving the significance of the decision tree.

In a further technically advantageous embodiment of the method, the image is evaluated by a user via an evaluating facility. This, for example, achieves the technical advantage that information on the image quality can be stored for the image.

In a further technically advantageous embodiment of the method, the data on the patient properties and the associated scan protocol of the evaluated image is used as a further training data set. This, for example, achieves the technical advantage that the amount of training data is increased thus improving the choice of scan protocol.

In a further technically advantageous embodiment of the method, the training data sets are achieved via a data interface from a central data store to which a plurality of imaging devices is connected. This, for example, achieves the technical advantage that it is possible to have recourse to a large database.

A second embodiment is directed to an imaging system for carrying out a medical examination with: a decision-tree creator for the creation of a decision tree on the basis of training data sets each of which comprises data on patient properties and an assigned scan protocol; a selecting facility for the selection of a scan protocol based on the decision tree created and a patient data set comprising data on the patient properties of the patient to be examined; and an imaging device for the creation of an image via the imaging device based on the selected scan protocol. The imaging system achieves the same technical advantages as those achieved by the method according to the first embodiment.

In one technically advantageous embodiment of the imaging system, the decision-tree creator is embodied to create the decision tree on the basis of a C4.5 algorithm and/or an ID3 algorithm.

In a further technically advantageous embodiment of the imaging system, the decision-tree creator is embodied to create the decision tree on the basis of the averaging of a plurality decision trees.

In a further technically advantageous embodiment of the imaging system, the imaging device comprises an evaluating facility for the evaluation of the image created by a user.

In a further technically advantageous embodiment of the imaging system, the decision-tree creator is embodied to use the data on the patient properties and the associated scan protocol of the evaluated image as a further training data set.

In a further technically advantageous embodiment of the imaging system, the imaging system comprises a data interface for retrieving the training data sets from a central data store to which a plurality of imaging devices is connected.

In a further technically advantageous embodiment of the imaging system, the imaging device is a computed tomography scanner or a magnetic resonance scanner.

A third embodiment is directed to a computer program product comprising software code sections, which can be loaded the directly into the memory of a digital computer with which the method according to the first embodiment is carried out when the software code sections are executed by the computer.

The computer program product can be formed by a computer program or comprise at least one additional component in addition to the computer program. The at least one additional component can be embodied as hardware and/or software.

One example of the at least one additional component, which is embodied as hardware, is a storage medium that can be read by the digital computer and/or on which the software code sections are stored.

One example of the at least one additional component, which is embodied as software, is a cloud application program embodied to divide the software code sections into different processing units, in particular different computers, of a cloud computing system, wherein each of the processing units is embodied to execute one or more software code sections.

In particular, the software code sections can be used to carry out the method according to the first aspect when the software code sections are executed by the processing units of the cloud computing system.

FIG. 1 is a schematic representation of an imaging system 100. The system comprises an imaging device 115, which creates an image 123 of a patient to be examined on the basis of a scan protocol. The scan protocol specifies the technical parameters for carrying out the imaging examination such as, for example, radiation doses or pulse lengths. These technical parameters are used by the imaging device 115 when carrying out the examination. The imaging device 115 is for example a magnetic resonance tomography scanner or a computed tomography scanner.

The imaging device 115 comprises a decision-tree creator 101, which is used for the creation of a decision tree 103 on the basis of training data sets. The training data sets each comprise data on the relevant patient properties and a scan protocol assigned to these patient properties. The decision-tree creator 101 uses a C4.5 algorithm for training with respect to decision trees for the automatic definition of scan parameters. The input data used is CT scanner operation by expert users without these experts having explicitly to disclose their knowledge.

The imaging device 115 also comprises a selecting facility 105 for the selection of the scan protocol 109 based on the decision tree 103 created and on the basis of a patient data set 111 comprising the data on the patient properties of the patient to be examined. The patient data set 111 can be input into the imaging device 115 manually by an operator.

Generally, the decision-tree creator 101 and the selecting facility 105 can be arranged not only inside the imaging device 115 but also on other locations of the imaging system 100. The decision-tree creator 101 and the selecting facility 105 can be implemented by a computer program or a digital electric circuit.

The imaging system 100 also comprises a display 121 for depicting the images obtained and the decision trees created, such as, for example, a flat screen. On the display 121, an evaluating facility 113 enabling the evaluation of the image created 123 by a user can be provided or depicted on the display 121. This makes it possible to create a further training data set 107 for the image 123 comprising data on the patient properties input, the scan protocol used and the evaluation of the image quality by a user.

The imaging device 115 also comprises a data interface 117 via which the training data can be downloaded from a central server. The data interface 117 also enables further training data sets 107 to be uploaded to the central server. The central server can provide the training data to a plurality of imaging devices 115.

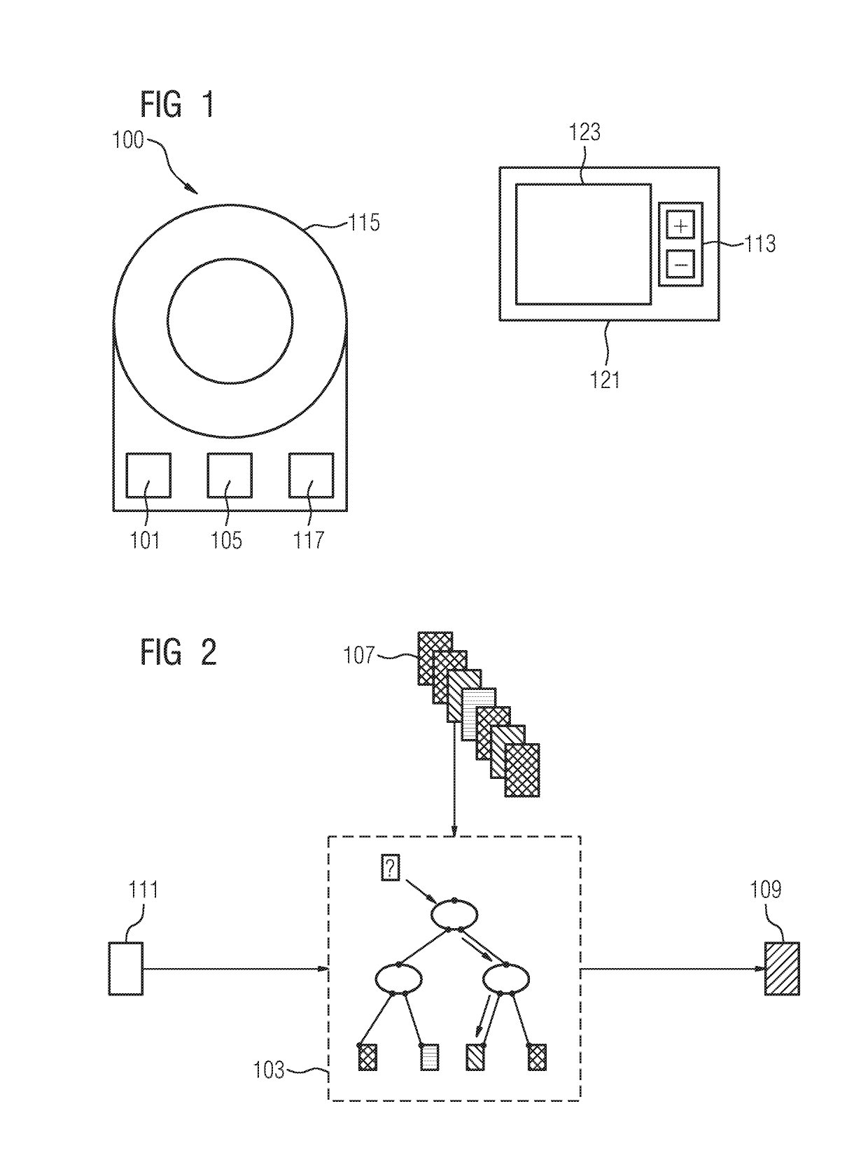

FIG. 2 shows in compressed form how the decision tree 103 is created and trained. First, the decision tree 103 is created on the basis of the training data sets 107. The training data sets 107 each comprise data on the patient properties, which is relevant for the examination on the imaging device 115, and an assigned scan protocol, with which high-quality images could be created in the past.

It is, for example, possible to use a C4.5 algorithm or an ID3 algorithm for the creation of the decision tree 103. The C4.5 algorithm is a concept-learning algorithm, i.e. a form of machine learning. The C4.5 algorithm is an extension of the ID3 algorithm. The C4.5 algorithm is used to create the decision tree 103 from the training data sets 107. The decision tree 103 created by automatic learning with the aid of the C4.5 algorithm can be displayed in a simple graphical form. It is also demonstrated that the decision on which the data is based is of low complexity and dependent upon the heart rate.

The C4.5 algorithm analyzes the data sets and arranges them according to maximum information content with respect to the present patient properties. This results in the creation of a decision tree 103 containing the most important decision-making criterion as a root for the decision. The relevance decreases during the further course of the decision tree 103. This has the advantage that complex rules are converted into a clearly intelligible decision tree 103.

The basic structure of an ID3 algorithm comprises the entry of the training data sets 107. If all the data sets of the training data sets 107 belong to the same class, a new leaf is created and marked with the respective class.

If not all the training data sets 107 belong to the same class, first, an attribute (property) is selected in accordance with a heuristic function. Then, a new node with the attribute is created as a test. Then, for each value of the attribute, the quantity of all data sets with values conforming to the attribute is determined, the ID3 algorithm is used to construct a decision tree 103 for the specific quantity and an edge is created which connects the nodes to the decision tree 103. Finally, the decision tree 103 created is output.

An information gain can be determined in that, when an attribute divides the training data sets 107 into subsets, the average entropy is calculated and the sum compared with the entropy of the original training data.

The information gain for an attribute A, the quantity S and the subsets Sᵢ is calculated as:

$$Gain ( S , A ) = E ( S ) - I ( S , A ) = E ( S ) - {\sum }_{i} \frac{\vert {S}_{i} \vert}{\vert S \vert} \cdot E ( {S}_{i} )$$

The attribute A chosen is that which maximizes the difference, i.e. the attribute that reduces the disorder to the greatest extent. In this case, maximization of the information gain is equivalent to minimization of the average entropy since E(S) is constant for all attributes A.

Then, a patient data set 111 is classified using the created decision tree 103 and assigned to a scan protocol 109. The patient data set 111 comprises data on patient properties, which are relevant for the examination on the imaging device 115, such as, for example, age, weight or the period for which the patient is able to hold breath. The patient data set 111 can be used to pass through the decision tree 103 starting from the root so that a scan protocol 109 to be used for the patient data set 111 is obtained at the leaves of the decision tree 103.

The scan protocol 109 selected in this way is used during the imaging examination with the imaging device 115.

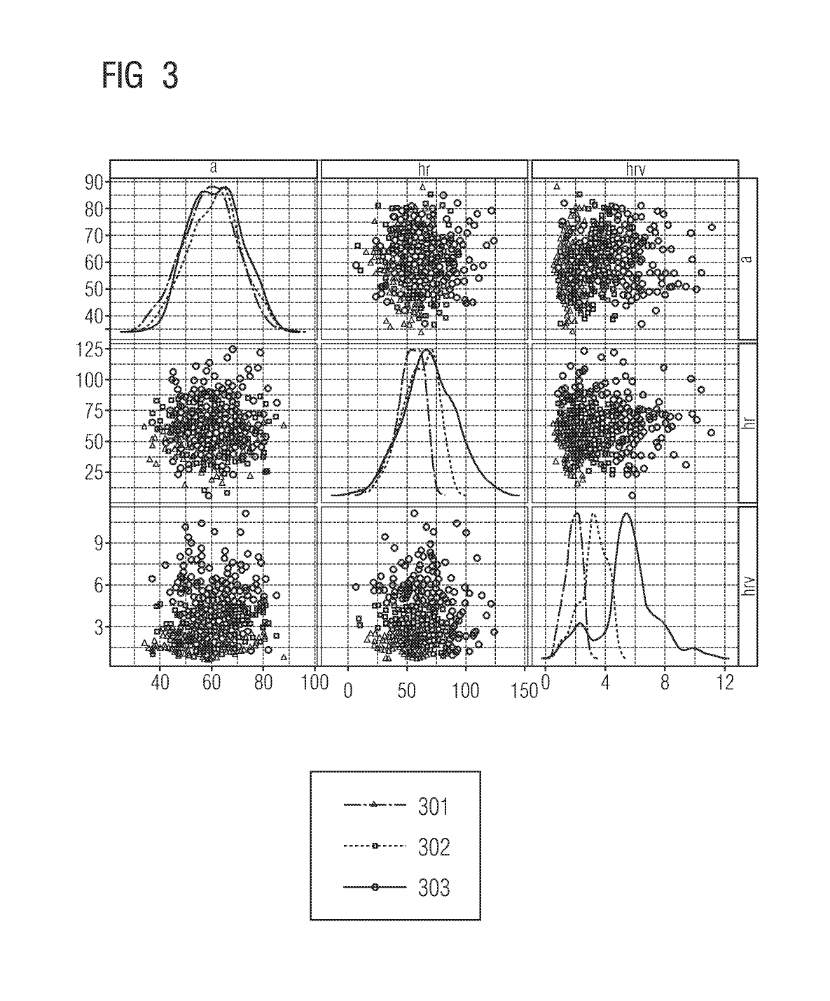

FIG. 3 shows a scatter plot. The data is based on a simulation depicting the relationship between the input variables and the scan protocol. In this case, the simulated behavior represents an expert with carefully considered decisions.

The relationship in the training data between the patient properties heart rate (hr), heart rate variability (hrv) and age (a) and the scan protocol target variable used in the learning (301—“high pitch”, 302—“sequence”, 303—“spiral”) is shown for a data set that is artificially diced-up by rules.

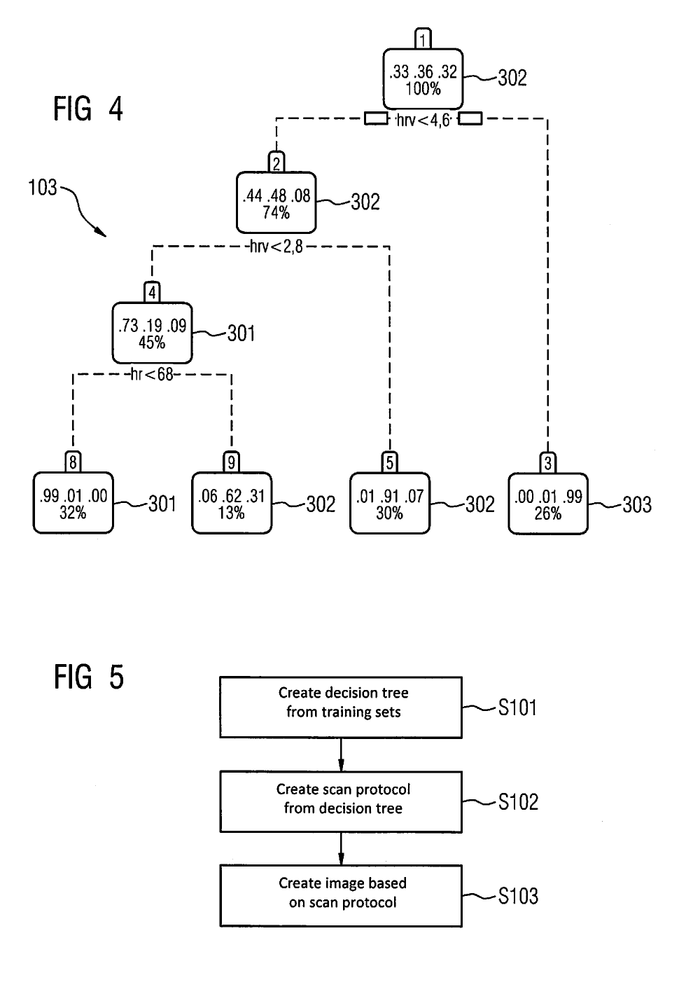

FIG. 4 shows the automatically created decision tree 103 for training data simulating expert knowledge. The decision tree 103 created by automatic learning with the aid of the C4.5 algorithm can be displayed in a simple graphical form. It is also demonstrated that the decision is primarily dependent upon the heart rhythm (hrv) and, to a secondary degree, on the average heart rate (hr). The accuracy is 92%, which is a measure of the consistency of the procedure.

By way of a behavior or user analysis based on training data sets 107, the decision-tree creator 101 provides a hierarchical decision tree 103 which arranges the individual decisions with respect to the scan protocol 109 according to their importance. This knowledge from the training data sets 107 is represented in the form of an automatically created decision tree 103. This is of advantage for convincing an operator of the accuracy and plausibility of the selected scan protocol 109 when using the imaging device 115. The intelligibility of the decision is based on the simple representation in the form of a decision tree 103 and in visual form as a sunburst diagram, which is derived from decision tree 103. The use of a modified boosting method also enables the integration in the algorithm of a simple feedback loop with respect to the scan result.

Simple representation as a decision tree 103 also enables manual adaptation of the decision tree 103 by shortening, extending or combining branches. The algorithm also makes it possible to deal with missing information on patient properties, such as, for example, if the weight of the patient is not known.

FIG. 5 is a block diagram of the method for carrying out the imaging medical examination. The method comprises the step of the creation S101 of the decision tree 103 on the basis of the training data sets 107 each of which comprises data on the relevant patient properties and an assigned scan protocol. In a step S102, a scan protocol 109 based on the decision tree 103 is created and the patient data set 111 is selected comprising data on the patient properties of the patient to be examined. In a step S103, the image is created via the imaging device 115 based on the selected scan protocol 109.

A database can comprise data from the behavior of recognized expert users from the respective clinical field as training data sets 107. In addition, the training of the decision tree 103 can be controlled in that it is determined from interactive feedback from the operators to the evaluating facility 113 whether the scan was successful (boosting). This enables a new training data set 107 to be included as a positive or negative example in the training with respect to the decision tree 103. In addition, it is also possible in a simple way to change and adapt the automatically generated decision trees 103. This property is of advantage for the maintenance and configuration of the automatic process when this is offered as a software element.

In this way, an automatically generated decision tree 103 can be used to select a scan protocol 109 based on an expert's rules and which, when used, results in intelligible decisions with respect to the scan protocol 109. In addition to a conventional tree representation, it is also possible to use a sunburst diagram to visualize the course of the procedure. It is also possible to average a plurality of decision trees 103 in order to be able to offer a solution for the selection of the scan protocol 109.

The type of data evaluation also enables the facility or Land-specific procedure to be analyzed with respect to the selection of the scan protocol and optionally forwarded as a further proposed solution within the framework of a transparent automatic procedure to other users. It is now possible to use a direct comparison to draw simple conclusions from the decision trees 103 created which enable the clinically permitted heart rate for a specific scan protocol to be determined in a simple way.

Also conceivable is a generalization for other decisions. Here, the basis and central step is the use of a dedicated decision tree algorithm, which uses the evaluation of existing data to provide a hierarchical decision tree that is intelligible to humans.

On the basis of the learning decision tree, an algorithm can analyze an operator's previous behavior and derive a new, optimal decision tree therefrom. The previous behavior is, for example, stored in a database with the scans already performed as training data sets 107 comprising the individual input criteria for the patient, such as, for example, weight, age, heart rate, rhythm and the scan protocol used in this case.

In addition, the combination of a trainings based on genuine data and the additional evaluation of the data with respect to the dose or time enables different strategies to be offered in a tree representation, which then generate a “low dose” or “night shift” tree for the same examination.

All the features explained and demonstrated in connection with individual embodiments of the invention can be provided different combinations in the subject matter according to the invention in order to achieve its advantageous effects simultaneously.

All the method steps can be implemented by devices suitable for carrying out the respective method step. All functions carried out by the features of the subject matter can be a method step of a method.

The scope of protection of the present invention is provided by the claims and is not restricted by the features explained in the description or figures.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. §112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A method for creating an image related to a patient to be examined during a medical examination, comprising:
creating of a decision tree based upon training data sets, each of the training data sets including data on patient properties and an assigned scan protocol, the decision tree being created based upon at least one of a C4.5 algorithm and an ID3 algorithm;
selecting a scan protocol based on the decision tree created and a patient data set including data on patient properties of the patient to be examined; and
creating the image, via an imaging device, based on the selected scan protocol.

2. (canceled)

3. The method of claim 1, wherein the decision tree is created based upon an averaging of a plurality of decision trees.

4. The method of claim 1, further comprising:
evaluating the image created by a user, via an evaluating facility.

5. The method of claim 4, wherein the data on the patient properties and the scan protocol for the evaluated image are used as a further training data set.

6. The method of claim 1, further comprising:
retrieving the training data sets, via a data interface, from a central data store to which a plurality of imaging devices are connected.

7. An imaging system for creating an image related to a patient to be examined during a medical examination, comprising:
a decision-tree creator to create a decision tree based upon training data sets, each of the training data sets including data on patient properties and an assigned scan protocol, the decision-tree creator being embodied to create the decision tree based upon at least one of a C4.5 algorithm and an ID3 algorithm;
a selecting facility to select a scan protocol based on the decision tree created and a patient data set including data on the patient properties of the patient to be examined; and
an imaging device to create an image based on the selected scan protocol.

8. (canceled)

9. The imaging system of claim 7, wherein the decision-tree creator is embodied to create the decision tree based upon averaging of a plurality of decision trees.

10. The imaging system of claim 7, wherein the imaging device includes an evaluating facility to evaluate the image created by a user.

11. The imaging system of claim 10, wherein the decision-tree creator is embodied to use the data on the patient properties and the scan protocol of the evaluated image as a further training data set.

12. The imaging system of claim 7, further comprising a data interface to retrieve the training data sets from a central data store to which a plurality of imaging devices is connected.

13. The imaging system of claim 7, wherein the imaging device is a computed tomography scanner or a magnetic resonance scanner.

14. A non-transitory computer program product comprising software code sections, directly loadable into the memory of a digital computer, to carry out the method of claim 1 when the software code sections are executed by the computer.

15. The method of claim 3, further comprising:
evaluating the image created by a user, via an evaluating facility.

16. The method of claim 15, wherein the data on the patient properties and the scan protocol for the evaluated image are used as a further training data set.

17. The method of claim 4, further comprising:
retrieving the training data sets, via a data interface, from a central data store to which a plurality of imaging devices are connected.

18. The method of claim 5, further comprising:
retrieving the training data sets, via a data interface, from a central data store to which a plurality of imaging devices are connected.

19. The method of claim 3, further comprising:
retrieving the training data sets, via a data interface, from a central data store to which a plurality of imaging devices are connected.

20. An imaging system for creating an image related to a patient to be examined during a medical examination, comprising:
an imaging device;
a first memory storing computer-readable instructions; and
one or more processors configured to execute computer-readable instructions to
create a decision tree based upon training data sets, each of the training data sets including data on patient properties and an assigned scan protocol, the decision tree being created based upon at least one of a C4.5 algorithm and an ID3 algorithm,
select a scan protocol based on the decision tree created and a patient data set including data on patient properties of the patient to be examined, and
create the image, via the imaging device, based on the selected scan protocol.

21. (canceled)

22. The imaging system of claim 20, wherein the decision-tree is created based upon averaging of a plurality of decision trees.

23. The imaging system of claim 20, wherein the imaging device includes an evaluating facility to evaluate the image created by a user.

24. The imaging system of claim 23, wherein the decision-tree is created using the data on the patient properties and the scan protocol of the evaluated image as a further training data set.

25. The imaging system of claim 20, further comprising
a data interface to retrieve the training data sets from a central data store to which a plurality of imaging devices is connected.

26. The imaging system of claim 20, wherein the imaging device is a computed tomography scanner or a magnetic resonance scanner.

27. A non-transitory computer readable including program sections, directly loadable into the memory of a digital computer, to carry out the method of claim 1 when the program sections are executed by the computer.
